(19) United States
(12) Patent Application Publication
(10) Pub. No.: US 2025/0166248 A1
(43) Pub. Date: May 22, 2025

(54) SYSTEMS AND METHODS FOR AUTOMATIC DISPLAY FIELD OF VIEW IN MEDICAL IMAGING

(71) Applicant: GE Precision Healthcare LLC, Waukesha, WI (US)

(72) Inventors: Eli Stern, Haifa (IL); Lilach Shay Levi, Haifa (IL); Gali Elkin-Basudo, Haifa (IL)

(21) Appl. No.: 18/517,813

(22) Filed: Nov. 22, 2023

(51) Int. Cl. G06T 11/00; A61B 6/00; G06T 7/12; G06T 7/62; G16H 10/60; G16H 30/40

(52) U.S. Cl. CPC G06T 11/005; A61B 6/461; A61B 6/488; A61B 6/5205; G06T 7/12; G06T 7/62; G06T 11/008; G16H 10/60; G16H 30/40; A61B 6/5294; G06T 2207/10081; G06T 2207/10104; G06T 2207/10108; G06T 2207/30168

(57) ABSTRACT

Methods and systems are herein provided for automatic determination of display field of view (DFOV) in medical imaging. In one example, a method comprises acquiring one or more scout images of a patient with an imaging system while the patient is positioned within a scanner of the imaging system; determining a body contour of the patient based on the one or more scout images; determining a widest dimension of the body contour; determining, based on the widest dimension, a display field of view (DFOV); acquiring scan data of the patient with the imaging system; reconstructing the scan data based on the DFOV to generate one or more reconstruction images; and displaying the one or more reconstruction images on a display device communicably coupled to the imaging system, wherein the patient remains within the scanner between acquisition of the one or more scout images and the scan data.

### FIELD

Embodiments of the subject matter disclosed herein relate to medical imaging, and more particularly to automatic determination of display field of view in medical imaging.

### BACKGROUND

Medical imaging systems may be used to capture images to assist a physician in making an accurate diagnosis. For example, a physician may use one or more images to visually identify a lesion or other anomalous structure in a patient. As another example, a physician may compare images taken over a series of patient visits to examine the evolution of a structure and/or to evaluate the effectiveness of a treatment. That is, the physician may examine morphological changes, such as changes in size and/or shape, of a lesion to evaluate its characteristics and/or the effectiveness of therapy.

Medical imaging systems may acquire raw data and then apply reconstruction algorithms to generate reconstructed images of the raw data. Raw data and reconstructed images are based on a variety of scanning parameters, scan protocols, and reconstruction parameters, all of which together inform image quality, target anatomy, pixel size, noise, and more, in the reconstructed images.

### BRIEF DESCRIPTION

In one example, a method comprises acquiring one or more scout images of a patient with an imaging system while the patient is positioned within a scanner of the imaging system; determining a body contour of the patient based on the one or more scout images; determining a widest dimension of the body contour; determining, based on the widest dimension, a display field of view (DFOV); acquiring scan data of the patient with the imaging system; reconstructing the scan data based on the DFOV to generate one or more reconstruction images; and displaying the one or more reconstruction images on a display device communicably coupled to the imaging system, wherein the patient remains within the scanner between acquisition of the one or more scout images and the scan data.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a pictorial view of a multi-modality imaging system according to an embodiment of the invention;

FIG. 2 is a block schematic diagram of an imaging system with a detector, according to an embodiment of the invention;

FIG. 3 is a block schematic diagram of a display field of view (DFOV) determination system of the imaging system of FIG. 2, according to an embodiment of the invention;

FIG. 4 is a flowchart illustrating a method for image acquisition with automatic DFOV determination.

FIG. 5 is a flowchart illustrating a method for determining a DFOV.

FIG. 6 shows an example image and corresponding body contour.

FIG. 7 shows example scout images of a patient.

FIG. 8 shows diagrams of a first image with pixels of a first size and a second image with pixels of a second size.

FIG. 9 shows a first example image reconstructed based on a default DFOV and a second example image reconstructed based on an estimated DFOV.

FIG. 10 shows a third example image reconstructed based on a default DFOV and a fourth example image reconstructed based on an automatically determined DFOV.

### DETAILED DESCRIPTION

This description and embodiments of the subject matter disclosed herein relate to methods and systems for automatically determining display field of view (DFOV) in medical imaging. In positron emission tomography (PET), computerized tomography (CT), PET-CT, and other types of imaging modalities, a scanner acquires images of patients based on one or more parameters, including the geometry of the scanner, type of scan, and the like. One of the parameters may be a scan field of view (SFOV), which determines the dimensions scanned. In most cases, the SFOV is greater than a widest dimension of the patient being imaged so as to acquire data of all relevant anatomy. Initial scan data may be a raw data of everything encompassed by the SFOV. DFOV may determine how much of the SFOV is reconstructed into an image.

In some examples, the SFOV is larger than the largest dimensions of the patient and as such, reconstructed images may contain data extraneous to the patient's body. Optionally, images may be reconstructed from the scan data using a smaller DFOV in order to reduce redundant areas from the image. Further, reducing DFOV compared to SFOV may also enable use of smaller virtual voxels, which may result in higher resolution images. For example, in PET-CT imaging image reconstruction is traditionally performed using 4 mm in-line pixel size (e.g., 4×4×4 mm voxels). However, this is a relatively large voxel size and may affect image quality by limiting the image spatial resolution. Poor spatial resolution additionally introduces partial-volume effect, which may negatively affect images both visually and quantitatively, resulting in decrease of signal in smaller lesions and image smoothing. Image quality in this manner may be increased by using a smaller in-line pixel size and consequently voxel size during reconstruction. However, reconstructing images with too small of a pixel size may result in increased noise. DFOV is directly related to pixel size, wherein pixel size is determined by converting the DFOV from cm to mm and then dividing by matrix size (e.g., two-dimensional (2D) grid of pixels) of the scan data.

In some cases, DFOV may be manually estimated and set to the scanner by a user based on a patient's body max index (BMI) or visual estimation of body size. In some examples, DFOV may be estimated in this manner prior to reconstructing images. In other examples, retrospective reconstruction performed with an estimated DFOV based on user assessment of an initial reconstructed image. In this way, the DFOV may be altered to fit an estimated size of the patient. However, BMI is an inaccurate measure of patient body contour due to varying degrees of tissue density (e.g., muscle vs fat). Estimating DFOV based on the BMI may provide a generalized DFOV, often erring towards a larger DFOV to avoid truncation of scan data. Estimation of DFOV in this manner may provide for less than ideal image resolution and may not remove all extraneous data from the reconstructed images.

Systems and methods are disclosed herein for automatically determining DFOV according to a patient body contour. Various medical imaging modalities, including PET-CT, among others, may utilize scout images for localizing the patient and a region of interest to be imaged. As an example, scout images may be used for correlating levels of axial images. As such, scout images show a body contour of the patient. By measuring a widest dimension of the patient's body contour within an acquired scout image, a DFOV specific to the scan of the patient may be automatically determined. In this way, the determined DFOV may be customized to the patient's body contour and may therefore allow for a pixel size that fits the patient's dimensions. The DFOV may be determined as any reasonable value less than the SFOV according to scan protocols and parameters. Determining DFOV based on the patient's specific body contour may allow for a more accurate DFOV used for reconstruction, leading to higher resolution images. Further, automatic determination of DFOV based on scout images may reduce time spent by the user in determining reconstruction parameters.

Further, automatically determining DFOV may be reversible. For example, in the event of excess noise levels or truncated edges in the reconstructed images, the scan data, which may be unaltered by reconstruction, may be reconstructed again based on a manually chosen DFOV, which may be different from the automatically determined DFOV, to reduce the noise or to result images that do not truncate the patient's body. In this way, DFOV may be flexible to meet scan parameters, image quality metrics, and patient dimensions.

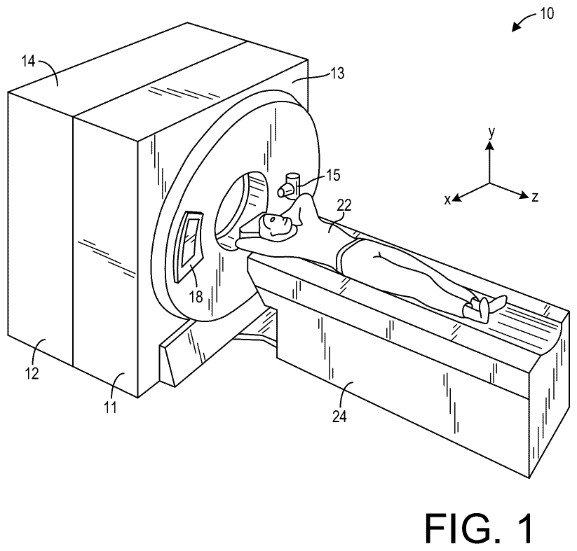

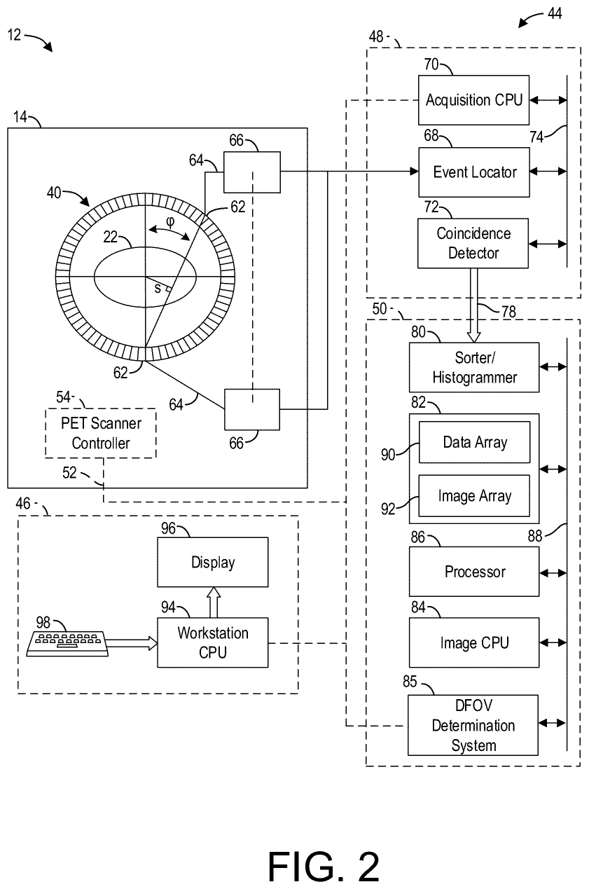

A multi-modality imaging system 10 is shown in FIGS. 1 and 2. Multi-modality imaging system 10 may be an example of an imaging system configured to acquire data of internal structures of a patient. Other types of imaging systems, such as a PET, a Single Photon Emission Computed Tomography (SPECT), SPECT-CT, or other system capable of generating tomographic images are possible without departing from the scope of this disclosure. The various embodiments are not limited to multi-modality medical imaging systems, but may be used on a single modality medical imaging system such as a stand-alone PET imaging system or a stand-alone SPECT imaging system, for example. Moreover, the various embodiments are not limited to medical imaging systems for imaging human subjects, but may include veterinary or non-medical systems for imaging non-human objects.

Referring to FIG. 1, the multi-modality imaging system 10 includes a first modality unit 11 and a second modality unit 12. The two modality units enable the multi-modality imaging system 10 to scan an object or patient in a second modality using the second modality unit 12. The multi-modality imaging system 10 allows for multiple scans in different modalities to facilitate an increased diagnostic capability over single-modality systems. In one embodiment, multi-modality imaging system 10 is a Computed Tomography/Positron Emission Tomography (CT/PET) imaging system 10, e.g., the first modality 11 is a CT imaging system 11 and the second modality 12 is a PET imaging system 12. The CT/PET system 10 is shown as including a gantry 13 representative of a CT imaging system and a gantry 14 that is associated with a PET imaging system. As discussed above, modalities other than CT and PET may be employed with the multi-modality imaging system 10.

The gantry 13 includes an x-ray source 15 that projects a beam of x-rays toward a detector array 18 on the opposite side of the gantry 13. Detector array 18 is formed by a plurality of detector rows (not shown) including a plurality of detector elements which together sense the projected x-rays that pass through a medical patient 22. Each detector element produces an electrical signal that represents the intensity of an impinging x-ray beam and hence allows estimation of the attenuation of the beam as it passes through the patient 22. During a scan to acquire x-ray projection data, gantry 13 and the components mounted thereon rotate about a center of rotation.

FIG. 2 is a block schematic diagram of the PET imaging system 12 illustrated in FIG. 1 in accordance with an embodiment of the present invention. The PET imaging system 12 includes a detector ring assembly 40 including a plurality of detector crystals. The PET imaging system 12 also includes a processor or controller 44, to control normalization, image reconstruction processes and perform calibration. Controller 44 is coupled to an operator workstation 46. Controller 44 includes a data acquisition processor 48 and an image reconstruction processor 50, which are interconnected via a communication link 52. PET imaging system 12 acquires scan data and transmits the data to data acquisition processor 48. The scanning operation is controlled from the operator workstation 46. The data acquired by the data acquisition processor 48 is reconstructed using the image reconstruction processor 50.

The detector ring assembly 40 includes a central opening, in which an object or patient, such as patient 22 may be positioned using, for example, a motorized table 24 (shown in FIG. 1). The motorized table 24 is aligned with the central axis of detector ring assembly 40. This motorized table 24 moves the patient 22 into the central opening of detector ring assembly 40 in response to one or more commands received from the operator workstation 46. A PET scanner controller 54, also referred to as the PET gantry controller, is provided (e.g., mounted) within PET system 12. The PET scanner controller 54 responds to the commands received from the operator workstation 46 through the communication link 52. Therefore, the scanning operation is controlled from the operator workstation 46 through PET scanner controller 54.

During operation, when a photon collides with a crystal 62 on a detector ring 40, it produces a scintillation event on the crystal. Each photomultiplier tube or photosensor produces an analog signal that is transmitted on communication line 64 when a scintillation event occurs. A set of acquisition circuits 66 is provided to receive these analog signals. Acquisition circuits 66 produce digital signals indicating the three-dimensional (3D) location and total energy of the event. The acquisition circuits 66 also produce an event detection pulse, which indicates the time or moment the scintillation event occurred. These digital signals are transmitted through a communication link, for example, a cable, to an event locator circuit 68 in the data acquisition processor 48.

The data acquisition processor 48 includes the event locator circuit 68, an acquisition CPU 70, and a coincidence detector 72. The data acquisition processor 48 periodically samples the signals produced by the acquisition circuits 66. The acquisition CPU 70 controls communications on a back-plane bus 74 and on the communication link 52. The event locator circuit 68 processes the information regarding each valid event and provides a set of digital numbers or values indicative of the detected event. For example, this information indicates when the event took place and the position of the scintillation crystal 62 that detected the event. An event data packet is communicated to the coincidence detector 72 through the back-plane bus 74. The coincidence detector 72 receives the event data packets from the event locator circuit 68 and determines if any two of the detected events are in coincidence. Coincidence is determined by a number of factors. First, the time markers in each event data packet must be within a predetermined time period, for example, 12.5 nanoseconds, of each other. Second, the line-of-response (LOR) formed by a straight line joining the two detectors that detect the coincidence event should pass through the field of view in the PET imaging system 12. Events that cannot be paired are discarded. Coincident event pairs are located and recorded as a coincidence data packet that is communicated through a physical communication link 78 to a sorter/histogrammer 80 in the image reconstruction processor 50. The image reconstruction processor 50 may store and/or employ one or more reconstruction algorithms, including maximum likelihood expectation maximization (MLEM)-based techniques such as ordered subset expectation maximization (OSEM), enhanced techniques such as block sequential regularized expectation maximization (BSREM), deep-learning techniques, and 3D iterative reconstruction algorithms.

The image reconstruction processor 50 includes the sorter/histogrammer 80. During operation, sorter/histogrammer 80 generates a data structure known as a histogram. A histogram includes a large number of cells, where each cell corresponds to a unique pair of detector crystals in the PET scanner. Because a PET scanner typically includes thousands of detector crystals, the histogram typically includes millions of cells. Each cell of the histogram also stores a count value representing the number of coincidence events detected by the pair of detector crystals for that cell during the scan. At the end of the scan, the data in the histogram is used to reconstruct an image of the patient. The completed histogram containing all the data from the scan is commonly referred to as a “result histogram.” The term “histogrammer” generally refers to the components of the scanner, e.g., processor and memory, which carry out the function of creating the histogram.

The image reconstruction processor 50 also includes a memory module 82, an image CPU 84, an array processor 86, and a communication bus 88. During operation, the sorter/histogrammer 80 counts all events occurring along each projection ray and organizes the events into 3D data. This 3D data, or sinogram, is organized in one exemplary embodiment as a data array 90. Data array 90 is stored in the memory module 82. The communication bus 88 is linked to the communication link 52 through the image CPU 84. The image CPU 84 controls communication through communication bus 88. The array processor 86 is also connected to the communication bus 88. The array processor 86 receives data array 90 as an input and reconstructs images in the form of image array 92. Resulting image arrays 92 are then stored in memory module 82.

The images stored in the image array 92 are communicated by the image CPU 84 to the operator workstation 46. The operator workstation 46 includes a CPU 94, a display 96, and an input device 98. The CPU 94 connects to communication link 52 and receives inputs, e.g., user commands, from the input device 98. The input device 98 may be, for example, a keyboard, mouse, a touch-screen panel, and/or a voice recognition system, and so on. Through input device 98 and associated control panel switches, the operator can control the operation of the PET imaging system 12 and the positioning of the patient 22 for a scan. Similarly, the operator can control the display of the resulting image on the display 96 and can perform image-enhancement functions using programs executed by the workstation CPU 94.

Additionally, as described in greater detail herein, PET imaging system 12, and specifically the image reconstruction processor 50, may include a DFOV determination system 85. The DFOV determination system 85 may analyze acquired scout images, determine dimensions of patient body contour based on the scout images, and determine a DFOV for the image reconstruction processor 50 to use when reconstructing images. Determination of DFOV may also be based on one or more scan protocols and parameters, such as type of scan (e.g., cardiac scans vs abdominal scans) and SFOV. Determined DFOV may be displayed to the operator via workstation CPU 94 and/or display 96. For example, a scout image of a patient may be taken, a largest dimension of the patient's body contour may be determined, and a DFOV may be determined based on the largest dimension, as will be further described below.

The detector ring assembly 40 includes a plurality of detector units. The detector unit may include a plurality of detectors, light guides, scintillation crystals and analog application specific integrated chips (ASICs). For example, the detector unit may include twelve SiPM devices, four light guides, 144 scintillation crystals, and two analog ASICs.

As described previously, it should be understood that while a PET-CT system is herein described with respect to FIGS. 1 and 2, automatic DFOV determination as herein presented may be implemented for other appropriate imaging systems.

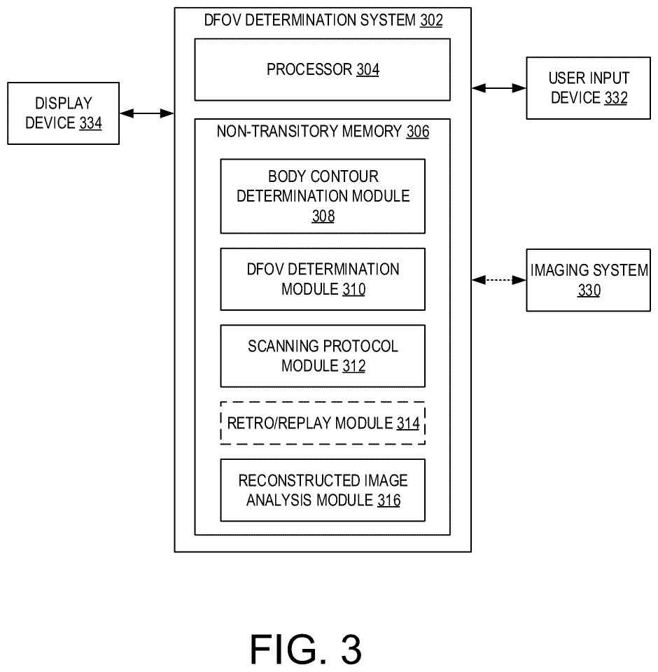

Referring now to FIG. 3, a diagram of a DFOV determination system 302 of a computing device is shown, in accordance with an embodiment, where DFOV determination system 302 may determine automatically a DFOV according to which scan data may be reconstructed. The DFOV determination system 302 may be employed to automatically determine DFOV during diagnostic image acquisition performed using an imaging system, such as PET imaging system 12 described above in reference to FIGS. 1 and 2. As such, DFOV determination system 302 may be a non-limiting example of DFOV determination system 85 of FIG. 2. In some examples, DFOV determination system 302 may be incorporated into the imaging system, as described above. For example, DFOV determination system 302 may rely on controller 44 and memory module 82. In some examples, at least a portion of DFOV determination system 302 is disposed at a device (e.g., workstation, edge device, server, etc.) communicably coupled to the imaging system via wired and/or wireless connections, which can receive images from the imaging system or from a storage device which stores the images/data generated by the imaging system. DFOV determination system 302 may be operably/communicatively coupled to a user input device 332 and a display device 334. User input device 332 may comprise the input device 98 of the PET imaging system 12, while display device 334 may comprise the display 96 of the PET imaging system 12, at least in some examples.

DFOV determination system 302 includes a processor 304 configured to execute machine readable instructions stored in non-transitory memory 306. Processor 304 may be single core or multi-core, and the programs executed thereon may be configured for parallel or distributed processing. In some examples, processor 304 may optionally include individual components distributed throughout two or more devices, which may be remotely located and/or configured for coordinated processing. In some examples, one or more aspects of processor 304 may be virtualized and executed by remotely-accessible networked computing devices configured in a cloud computing configuration.

Non-transitory memory 306 may store a body contour determination module 308, a DFOV determination module 310, a scanning protocol module 312, in some examples, a retro/replay module 314, and a reconstructed image analysis module 316. Each of the modules herein described may store data, including instructions executable by the processor 304. For example, the body contour determination module 308 may include instructions for generating a simple mask, e.g., via a segmentation algorithm, to define a contour map. A stored algorithm may be executed to analyze the contour map in order to determine a largest diameter. The analysis may be performed for a diameter of a largest circle positioned around an isocenter within an axial plane, as will be further described. The isocenter may be a 3D center point of the gantry that the tube and detector rotate around, therefore the scan data may be centered around the isocenter. The largest dimension may be measured in centimeters, millimeters, pixels, or other unit as set by the imaging system.

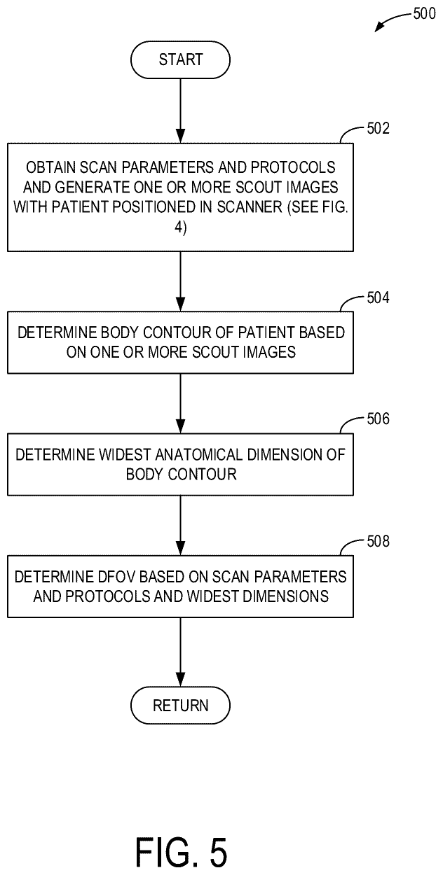

The DFOV determination module 310 may store instructions executable to determine a DFOV for reconstructing the scan data. The DFOV may define how much of the SFOV is reconstructed into an image. A maximum DFOV may be equal to the SFOV. DFOV may be determined based on the largest dimension of the patient body contour as determined via the body contour determination module 308 based on one or more scout images, as will be described further with respect to FIG. 5.

The scanning protocol module 312 may store various scanning protocols and parameters, for example in a look up table. As an example, each scanning protocol may have a set SFOV. The DFOV determination module 310 may take into account the scanning protocols stored in scanning protocol module 312 when determining DFOV. As noted, the maximum DFOV may be equal to the SFOV and therefore when determining DFOV, the SFOV, as stored within the scanning protocol module 312, may be obtained to inform DFOV determination.

In some examples, the DFOV determination system 302 may comprise the retro/replay module 314. In other examples, a retro/replay module may be included in the imaging system 330, such as multi-modality imaging system 10. The retro/replay module 314, and other retro/replay modules, may store one or more retrospective reconstruction algorithms for reconstructing scan data based on a different, in some instances manually inputted, DFOV.

Additionally, in some examples, the DFOV determination system 302, via the reconstructed image analysis module 316, may analyze the reconstructed images to determine noise level and analyze the edges to ensure that the data of the patient body is not truncated. If a determined noise level is above a predefined threshold and/or one or more edges are truncated, a prompt may be displayed on the display device 334 indicating to the operator that retrospective reconstruction based on a different DFOV is recommended.

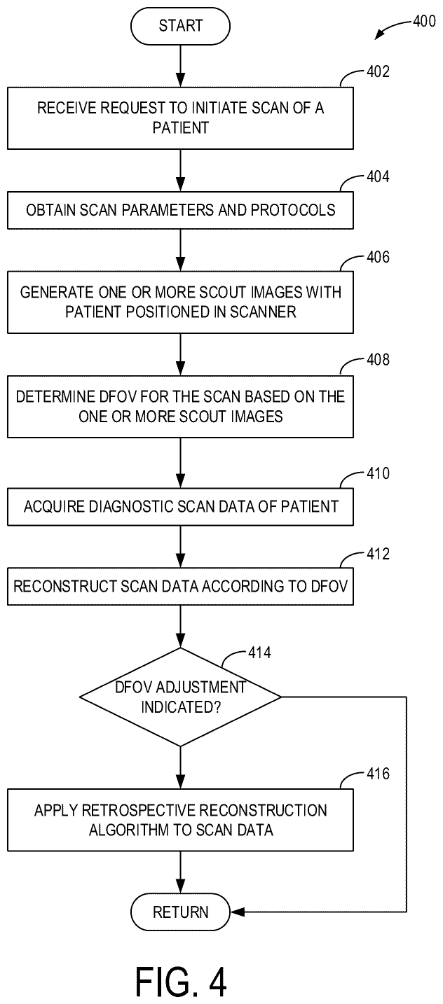

Turning now to FIG. 4, a flowchart illustrating a method 400 for image acquisition with automatic DFOV sizing is shown. Method 400 may be carried out using the systems and components described herein above with regards to FIGS. 1-3, though it should be understood that the method 400 may be carried out using other imaging systems and computing devices configured to acquire scout images and scan data and reconstruct the scan data into diagnostic images. For example, method 400 may be carried out according to instructions stored in memory of one or more controllers, processors, and/or computing devices included as part of and/or communicatively or operatively coupled to an imaging system, such as image reconstruction processor 50 of PET imaging system 12 of multi-modality imaging system 10 described with reference to FIGS. 1-2 and/or the DFOV determination system 302 described with respect to FIG. 3.

At 402, method 400 includes receiving a request to initiate a scan of a patient. The request may be a user selection or other user input inputted by a user via a user device (e.g., input device 98 of FIG. 1) in communication with an imaging system, such as PET imaging system 12 of multi-modality imaging system 10 of FIG. 1. The request to initiate the scan of the patient may include indication of scan protocol, which may include protocols such as type of scan, target anatomy, etc.

At 404, method 400 includes obtaining scan parameters and protocol(s) based on the scan request. As noted, the request to initiate the scan may include indication of scan protocol. The scan protocol may indicate various scan parameters, including SFOV, target anatomy, etc. Other scan parameters, including bore size, maximal axial field of view, geometry of the scanner, and the like, may be obtained from the imaging system. The SFOV may be specific to the type of scan, location, and the like, and may be stored in memory of the imaging system in one or more units (e.g., cm, mm, pixels, etc.).

At 406, method 400 includes generating one or more scout images with the patient positioned within the scanner. Following positioning of the patient within the scanner and initiation of the scan, one or more scout images may be obtained. The one or more scout images may comprise an axial, lateral, and/or helical low-dose scan of the patient and the scout data may be used for various tasks, including determining a patient body contour, anatomy localization, etc., as non-limiting examples. The one or more scout images may be generated relatively quickly and may not demand diagnostic quality as compared to diagnostic imaging data (e.g., scan data).

The one or more scout images may be generated of the same target anatomy as is the scan data is to be acquired. In instances in which contrast, a radioactive tracer, or other injectable material is to be injected into the patient, the scout images may be generated prior to or following injection, depending on the type of injectable material. For example, scout images may be generated following injection of a radioactive tracer but prior to full uptake. As another example, scout images may be generated prior to injection of a contrast agent (e.g., non-contrast scout images). In some examples, the one or more scout images may be 2D. In other examples, the one or more scout images may be 3D.

At 408, method 400 includes determining a DFOV for the scan based on the one or more scout images. The DFOV may be determined based on a body contour of the patient as determined and analyzed based on the one or more scout images, as will be further described with respect to FIG. 5. The DFOV may be determined while the patient is positioned within the scanner. The DFOV may be determined in the same units as the SFOV and as the body contour of the patient. The determined DFOV may be set to the scanner for use for acquired diagnostic imaging data.

At 410, method 400 includes acquiring diagnostic scan data of the patient. The diagnostic scan data may be acquired according to scan parameters and protocols as previously determined and/or obtained (e.g., during request or previous to the request). Acquisition of diagnostic scan data may include acquiring scan data of the patient, for example of a particular target anatomy or region based on a scan type. In some examples, the diagnostic scan data may include data of the same region of the patient imaged in the one or more scout images. In some examples, diagnostic scan data may be acquired immediately following determination of DFOV size. In other examples, diagnostic scan data may be acquired at the same time, or otherwise at overlapping times, as the DFOV is being determined. As such, the patient may remain within the scanner throughout, reducing time spent for the operator and the patient during the scan session. The scan data may be stored in memory of the imaging system.

At 412, method 400 includes reconstructing the scan data according to the DFOV. At least a portion of the scan data may be reconstructed into one or more images. The data reconstructed may be data within a scan field based on the DFOV. In some examples, the DFOV may be equal to the SFOV, and therefore all the scan data may be reconstructed. In other examples, the DFOV may be smaller than the SFOV and therefore a portion of the data may be reconstructed. As is described with reference to FIG. 2, the data array may be reconstructed into one or more images by a reconstruction processor according to a reconstruction algorithm. The one or more images may be 3D or a collection of 2D images, depending on the type of imaging system used. The one or more images may be stored as an image array in memory and may be displayed on a display device for visualization by the user.

At 414, method 400 includes judging whether DFOV adjustment is indicated. As described with respect to FIG. 3, reconstructing scan data based on a DFOV that is smaller than the SFOV may result in smaller voxels and therefore higher resolution, but in some instances, smaller voxels may result in higher noise in the reconstructed images. In some examples, noise above a predefined threshold may be detected by the imaging system (e.g., via the reconstructed image analysis module 316) and a notification may be outputted on the display device indicating a recommendation for DFOV adjustment. In other examples, determination that DFOV adjustment is indicated based on the amount of noise may be made by the operator.

Further, in some examples, the DFOV may result in truncation of portions of the scan data that correspond to patient anatomy. As described with respect to FIG. 3, in some examples, edges within the reconstructed images may be analyzed by the computing device to detect truncation of scan data corresponding to patient anatomy. Similar to as described above, a notification may be displayed on the display device indicating that DFOV adjustment is recommended on the basis of the truncation. In other examples, truncation of scan data in the images may be determined by the operator.

If DFOV adjustment is indicated based on noise, truncation, and/or other factor (YES at 414), method 400 proceeds to 416. If DFOV adjustment is not indicated based on noise, truncation, and/or other factor (NO at 414), method 400 ends.

At 416, method 400 includes applying retrospective reconstruction to the scan data to generate reconstructed images. As noted above, the scan data may be stored in memory and may be unaffected by initial reconstruction. Therefore, retrospective reconstruction may be applied to reconstruct the scan data based on a different DFOV than the DFOV determined at 408. In some examples, the different DFOV may be determined by manual input from the operator and may be larger than the automatically determined DFOV. In other examples, retrospective reconstruction may be performed based on a second automatically determined. In this way, reconstruction may be repeatable for one or more second DFOVs to generate one or more sets of reconstructed images additional to the originally generated reconstructed images. The newly reconstructed images may be displayed on the display device in a similar fashion to the originally reconstructed images.

As will be further described with respect to FIG. 5, determining DFOV based on the patient's body contour may provide a highly customized DFOV, which may allow for generation of reconstructed images with higher resolution than reconstructed images based on a default DFOV or an estimated DFOV (e.g., based on patient BMI) because the DFOV may be set to fit the patient's widest dimensions. Further, by automatically determining DFOV and reconstructing images based on the determined DFOV, time spent in repeating reconstructions with smaller DFOVs following acquisition may be reduced, saving time for the operator in performing the scan and saving time for the patient in waiting to receive results.

Additionally, automatically determining a DFOV that is specific to the scan and the patient may increase processing efficiency. In some examples, customizing the DFOV as herein described may reduce and/or entirely mitigate demand for repeated reconstructions with different DFOVs, thereby reducing processing power demanded by repeat reconstruction. Further, the method takes advantage of scout images, which may be obtained as part of a typical data acquisition protocol, and thereby allows for decreased overall processing power as a simple segmentation is performed in order to reduce repeated reconstruction of acquired diagnostic data.

Turning now to FIG. 5, a flowchart illustrating a method 500 for automatically determining DFOV based on a patient body contour is shown. Method 500 may be carried out using the systems and components described herein above with regards to FIGS. 1-3, though it should be understood that the method 500 may be carried out using other imaging systems configured to acquire scout images and scan data and reconstruct the scan data into diagnostic images. For example, method 500 may be carried out according to instructions stored in memory of one or more controllers, processors, and/or computing devices included as part of and/or communicatively or operatively coupled to an imaging system, such as image reconstruction processor 50 of PET imaging system 12 of multi-modality imaging system 10 described with reference to FIGS. 1-2 and/or the DFOV determination system 302 described with respect to FIG. 3. In some examples, the method 500 may be part of the method 400 described with respect to FIG. 4.

At 502, method 500 includes obtaining scan parameters and protocols and generating one or more scout images with the patient positioned in the scanner. As is described with respect to FIG. 4, one or more scan protocols and parameters, including type of scan and SFOV, may be obtained from the scanner and/or from a request to initiate a scan by an operator. One or more scout images may be obtained based on the one or more scan protocols and parameters, also as is described with respect to FIG. 4. The one or more scout images may be generated of the same field of view as the diagnostic scan data will be (e.g., of the same region of the body, with the same scan range, etc.).

At 504, method 500 includes determining a body contour of the patient based on the one or more scout images. In some examples, the imaging system may define what is body and what is not body in the scout images of the patient. This may be performed via generation of a simple segmentation mask via a segmentation algorithm to define a contour map. The contour map may define edges of the patient's body for the portion of the patient's body that is imaged in the one or more scout images. In this way, actual dimensions of the patient's body contour may be defined once the system determines what of the scout images comprises the patient's body and may be analyzed to determine widest/largest dimensions of the patient's body.

At 506, method 500 includes determining a widest anatomical dimension of the body contour. As described previously, determining the body contour of the patient may comprise determining a widest dimension of the patient. The widest dimension of the patient may be measured as a largest diameter of a circle positioned about an isocenter of the scanner. The largest diameter may be oriented along any line within an axial plane. As such, the widest dimension may inform largest dimension of the contour map among lateral and anterior-posterior (AP) data. In some examples, two or more regions of the body may be considered for the widest dimension. As an example, a shoulder to shoulder lateral region, a hip to hip lateral region, an AP chest region, and an AP abdomen region may be considered in an algorithm for determining widest dimension. The two or more regions may be regions that are traditionally the widest or otherwise largest dimensions parts of the body amongst a variety of patients.

The algorithm may scan the contour map, or in some examples the two or more regions of the contour map, as is described above, to measure the dimensions and therefore determine the widest dimension of the patient. The widest dimension of the patient as herein determined may be measured in the same unit as the SFOV and the DFOV, e.g., cm, mm, pixels, etc. In this way, a direct correlation may be made between the widest dimension and the DFOV.

At 508, method 500 includes determining DFOV based on the scan parameters and protocols and the determined widest dimension of the patient. In some examples, a default DFOV may be set as one of the scan parameters based on the SFOV and the scan type. For example, the default DFOV for a whole body PET-CT scan may be 70 cm while the default DFOV for a cardiac PET-CT may be 50 cm due to target imaging region/anatomy differences. The automatically determined DFOV may be equal to the widest dimension of the patient±a predefined margin (e.g., 2-3 cm). In some examples, the predefined margin may be added to the widest dimension to determine the DFOV to mitigate truncation of scan data in the reconstructed images. The determined DFOV may therefore be customized and specific to the scan of the patient as it may be any reasonable value within technical limitations (e.g., no larger than the SFOV), rather than an estimated value as may be determined manually.

As is described with respect to FIG. 4, the determined DFOV may be used when reconstructing the scan data into reconstructed images. The determined DFOV may be saved to memory and displayed as annotation data along with the reconstructed images by a display device. The scan data may be saved separate from the determined DFOV and the reconstructed images and may be unaltered by reconstruction, as previously discussed.

In this way, the determined DFOV may be customized specifically for the patient based on their body contour, dimensions, and the scan parameters/protocols set for the scanner. The customized DFOV may allow for higher resolution reconstructed images while mitigating generation of excess noise that may result from too small of voxels.

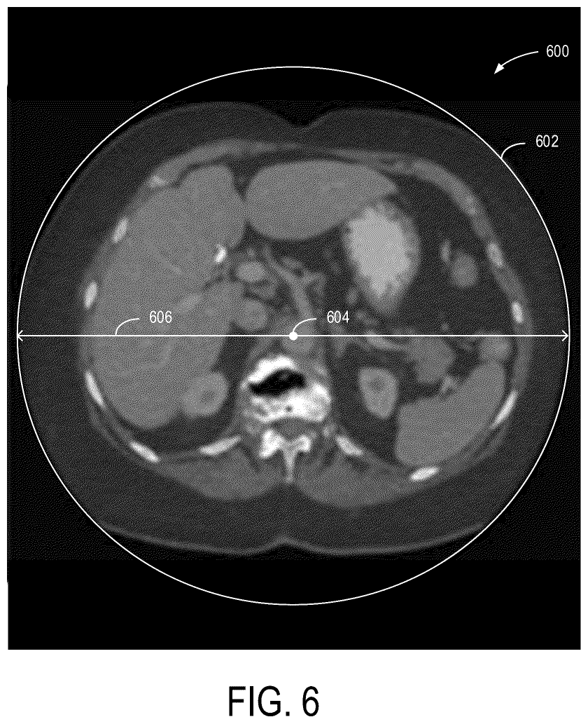

Referring now to FIG. 6, an example medical image 600 is shown. In some examples, the medical image 600 may be a scout image acquired by an imaging system configured to acquire both scout images and diagnostic scan data. In other examples, the medical image 600 may be an image of a patient or other subject reconstructed from scan data acquired by an imaging system configured to obtain scout images and diagnostic scan data and to reconstruct the diagnostic scan data into one or more reconstructed images. For example, the imaging system may be the multi-modality imaging system 10 described with respect to FIG. 1.

As is discussed herein, reconstruction may be based on a DFOV, which defines a pixel size for reconstruction. The DFOV may be a default value, an estimated value (e.g., based on patient BMI) or, as is presented herein, may be automatically determined based on the patient's body contour as determined based on scout images acquired by the imaging system, as described with respect to FIGS. 4-5.

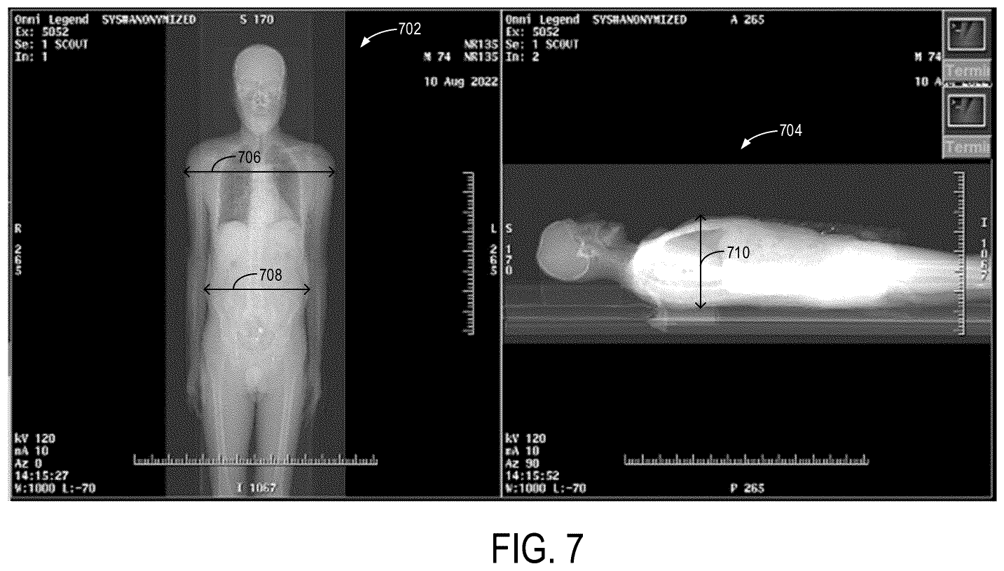

The medical image 600 as shown may demonstrate a widest dimension of the patient's body contour. The widest dimension of the patient's body contour may be determined from a patient's whole body or from two or more regions within one or more scout images, as will be described with respect to FIG. 7. In some examples, the medical image 600 may be a single axial image slice. Within the medical image 600, a line 606 corresponds to the widest dimension of the patient's body contour. The line 606 may be a diameter of a circle 602 positioned about an isocenter 604 and as such the line 606 may intersect the isocenter 604. The circle 602 may be the largest circle possible for the image where two opposing points of the circle 602 contact the patient body data within the medical image 600, wherein the two opposing points are end points of the line 606. A length of the line may therefore be the widest dimension of the patient body contour and may be used to determine a DFOV for the patient.

FIG. 7 shows one or more scout images that may be acquired prior to diagnostic scan data. The one or more scout images may comprise a first scout image 702 and a second scout image 704 acquired by an imaging system, such as the multi-modality imaging system 10 of FIG. 1.

In some examples, the first scout image 702 may be an AP image and the second scout image 704 may be a lateral image. A plurality of lines may be overlaid on the first and second scout images 702, 704, including a first line 706, a second line 708, and a third line 710. In some examples, each of the first, second, and third lines 706, 708, 710 may correspond to a specified region of the patient's body, or a contour map generated based on the one or more scout images, that is to be considered when determining a widest dimension of the patient body contour.

For example, the first line 706 may correspond to a shoulder to shoulder lateral region, the second line 708 may correspond to a hip to hip lateral region, and the third line 710 may correspond to a chest AP region. The shoulder to shoulder lateral region, hip to hip lateral region, and chest AP region, among others, may be defined as regions that typically include the widest dimension of a patient. Each of the regions may define an area covering a predefined distance along the axial axis (e.g., a z-axis). An algorithm to determine widest dimension of the patient may scan each of the regions to find the widest dimension.

As an example, the first line 706 may correspond to a widest dimension of the shoulder to shoulder lateral region, the second line 708 may correspond to a widest dimension of the hip to hip lateral region, and the third line 710 may correspond to a widest dimension of the chest AP region. Each line may have a length, for example in cm, and the length of each of the lines may be compared to each other to determine the longest. For example, the second line 708 may be longer than the third line 710, indicating that the hip to hip lateral region is wider than the chest AP region, and the first line 706 may be longer than the second line 708, indicating that the shoulder to shoulder lateral region is wider than both the hip to hip lateral region and the chest AP region. As is described with respect to FIG. 6, each of the lines may be diameters of respective circles positioned around respective isocenters for particular slices.

In other examples, the patient's whole body, in the form of a contour map as previously described, may be scanned by the algorithm to find the widest dimension. The first, second, and third lines 706, 708, 710 may be examples of such dimensions, with the first line 706 being determined to be the widest dimension of the patient. The widest dimension of the patient, as herein determined, may be used to define a DFOV for the patient such that reconstructed images based on the DFOV may contain all data relevant to the patient body while avoiding reconstructing data extraneous to the patient body.

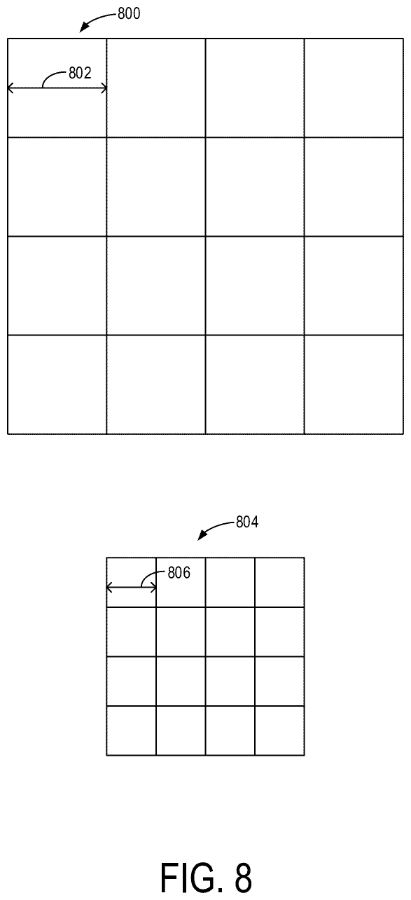

As is described above, DFOV may be directly related to pixel size, wherein smaller DFOV results in smaller pixel and voxel size. FIG. 8 is a block diagram of a first image 800 with pixels of a first size 802 and a second image 804 with pixels of a second size 806. Each of the first image 800 and the second image 804 may have the same matrix size. While a matrix size of 4 pixels is shown in FIG. 8, it should be understood that matrix sizes may vary, with a usual matrix size being 512 pixels. The first image 800 may correspond to a first DFOV and the second image 804 may correspond to a second DFOV. The second size 806 of the pixels of the second image 804 may be smaller than the first size 802 of the pixels of the first image 800.

Pixel size may be calculated by equation (1):

$$P S = \frac{{DFOV}_{m m}}{M S} \qquad ( 1 )$$

where PS is pixel size, MS is matrix size, and DFOVₘₘ is DFOV in millimeters.

As an example, the first image 800 may be reconstructed based on a DFOV of 70, which in some examples may be a default DFOV, while the second image 804 may be reconstructed based on a DFOV of 50. With a standard matrix size of 512, the DFOV of 70 may yield a pixel size of 1.37 mm for the first size 802 and the DFOV of 50 may yield a pixel size of 0.98 mm for the second size 806. As described above, smaller pixel sizes may result in higher resolution images and as such, reducing DFOV automatically as much as is reasonable based on the patient's body contour may result in higher resolution images.

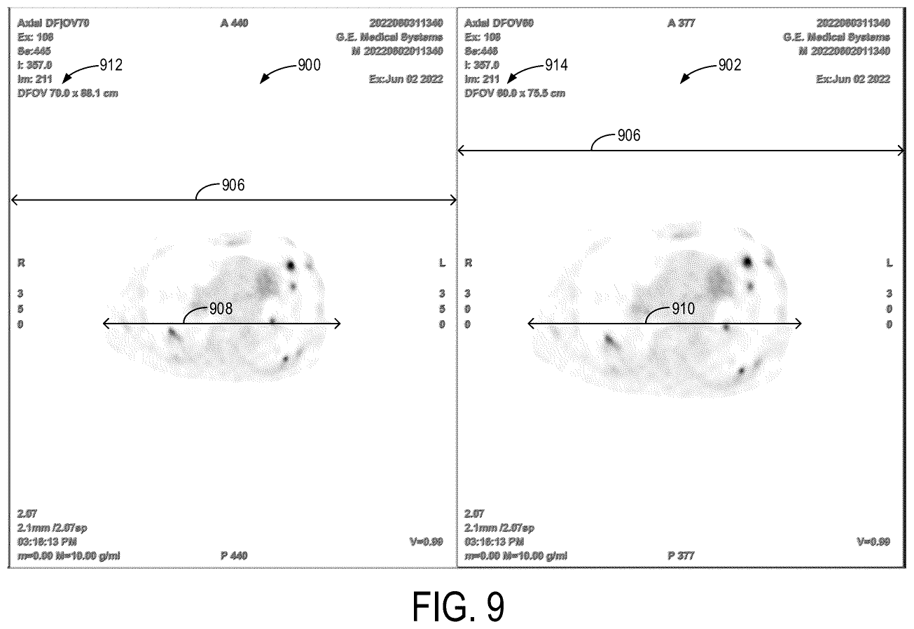

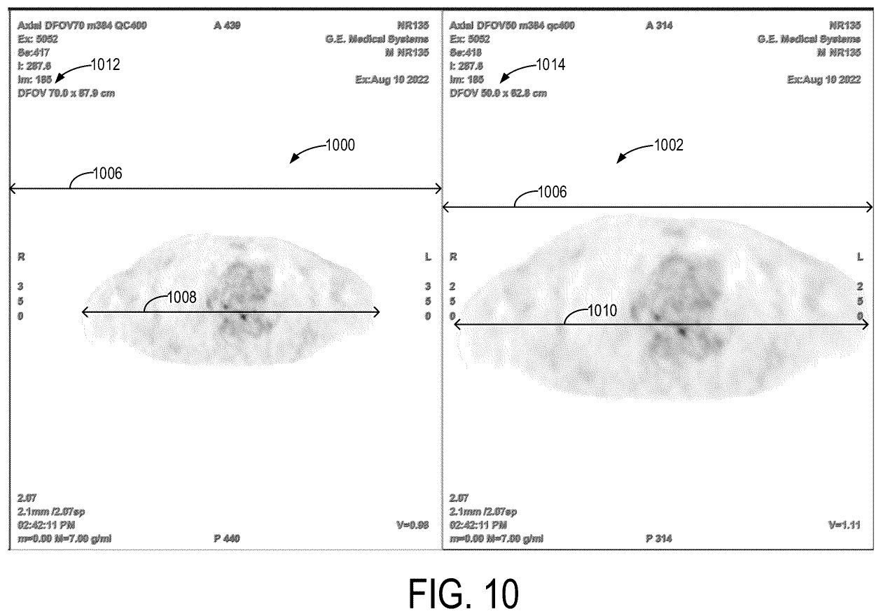

FIGS. 9 and 10 show examples of reconstructed images based on various DFOVs. Specifically, FIG. 9 shows a first reconstructed image 900 of a first patient reconstructed based a first DFOV 912, shown on the left, and a second reconstructed image 902 of the first patient reconstructed based on a second DFOV 914, shown on the right.

The first and second reconstructed images 900, 902 may be reconstructed from the same scan data, in some examples. The first DFOV 912 may be a default DFOV and the second DFOV 914 may be an estimated DFOV based on the first patient's BMI. A width 908 of the first patient's body within the first reconstructed image 900 may be smaller than a width 906 of the reconstructed image window. In some examples the width 906 of the reconstructed image window may be a parameter of the scanner/imaging system (e.g., of a display device of the imaging system, of a display device coupled to the imaging system) and may therefore be the same for the first reconstructed image 900 and the second reconstructed image 902 when acquired by the same scanner and/or reconstructed from the same scan data. In other examples, a width of the patient body within the reconstructed image window, e.g., the width 908, may be proportional to the width 906 of the reconstructed image window such that the width of the patient body from images acquired with the imaging system changes proportionally to the reconstructed image window width when the window size is changed.

The second DFOV 914 may be estimated based on the first patient's BMI. As an example, the first DFOV 912 may be 70 cm and the second DFOV 914 may be 60 cm. The second DFOV 914, being smaller than the first DFOV 912, may result in smaller voxels/pixels than the first DFOV 912. A width 910 of the first patient's body within the second reconstructed image 902 may be greater than the width 908 of the first patient's body within the first reconstructed image 900, but may still be less than the width 906 of the reconstructed image window. As such, the estimated DFOV may not removal all extraneous scan data from the reconstructed images.

FIG. 10 shows a third reconstructed image 1000 of a second patient reconstructed based on a third DFOV 1012, shown on the left, and a fourth reconstructed image 1002 of the second patient reconstructed based on a fourth DFOV 1014, shown on the right.

The third and fourth reconstructed images 1000, 1002 may be reconstructed from the same scan data, in some examples. As noted, the third reconstructed image 1000 may be a reconstructed image based on the third DFOV 1012. The third DFOV 1012 may be a default DFOV similar to the first DFOV 912. The fourth image 1002 may be a reconstructed image based on the fourth DFOV 1014. The fourth DFOV 1014 may be a DFOV determined based on the second patient's body contour, for example according to the methods described with respect to FIGS. 4-5. A width 1008 of the second patient's body within the third reconstructed image 1000 may be less than a width 1006 of the reconstructed image window, similar to as described with respect to the first reconstructed image 900. A width 1010 of the second patient's body within the fourth reconstructed image 1002 may be nearly equal to (e.g., +a predefined amount in cm, as previously described) the width 1006 of the reconstructed image window. The width 1010 may be nearly equal to the width 1006 because the fourth DFOV 1014 may be determined based on the second patient's body contour.

In this way, determining DFOV based on a patient's widest dimension of their body contour may allow for reduction of redundant areas from reconstructed images. Further, smaller DFOVs, as would allow for reduction of redundant areas, may increase reconstructed image resolution.

A technical effect of the systems and methods herein described is that image resolution may be increased and redundant areas of reconstructed images may be mitigated. Determining DFOV based on patient body contour dimensions may allow for a customized DFOV for a patient. Further, automated determination of the DFOV based on acquired scout images may reduce time spent by the operator by mitigating any time used to manually pick or estimate a DFOV, for example based on a BMI, as the DFOV is determined while the patient is positioned within the scanner and may be performed during normal scan time. Additionally, determining DFOV as herein described may be adjustable and/or reversible in the event of excess image noise or data truncation.

Further, as the determined DFOV is specific to the scan and patient, the demand for repeated reconstructions to optimize reconstructed images based on size within the window and resolution may be mitigated. Therefore, processing efficiency may be increased as reconstruction of data may need only be performed once.

The disclosure also provides support for a method, comprising: acquiring one or more scout images of a patient with an imaging system while the patient is positioned within a scanner of the imaging system, determining a body contour of the patient based on the one or more scout images, determining a widest dimension of the body contour, determining, based on the widest dimension, a display field of view (DFOV), acquiring scan data of the patient with the imaging system, reconstructing the scan data based on the DFOV to generate one or more reconstructed images, and displaying the one or more reconstructed images on a display device communicably coupled to the imaging system, wherein the patient remains within the scanner between acquisition of the one or more scout images and the scan data. In a first example of the method, determining the body contour of the patient comprises applying a segmentation mask to generate a contour map. In a second example of the method, optionally including the first example, determining the widest dimension of the body contour comprises scanning two or more regions of the contour map. In a third example of the method, optionally including one or both of the first and second examples, determining the DFOV is further based on one or more scan protocols and one or more scan parameters. In a fourth example of the method, optionally including one or more or each of the first through third examples, the one or more scan protocols comprise a scan type and a scan range. In a fifth example of the method, optionally including one or more or each of the first through fourth examples, the one or more scan parameters comprise a scan field of view (SFOV) and bore size. In a sixth example of the method, optionally including one or more or each of the first through fifth examples, the DFOV is directly related to pixel size of the one or more reconstructed images. In a seventh example of the method, optionally including one or more or each of the first through sixth examples, the method further comprises: applying a retrospective reconstruction algorithm to the scan data based on a second DFOV to generate one or more second reconstructed images in response to detection of one of excess noise and truncated data.

The disclosure also provides support for a system, comprising: a computing device communicatively coupled to an imaging system configured to image a patient, the computing device configured with instructions in non-transitory memory that when executed cause the computing device to: obtain one or more scan protocols and parameters for a requested scan of the patient, obtain one or more scout images of the patient according to the one or more scan protocols and parameters, generate a contour map of a body contour of the patient based on the one or more scout images, determine, based on the contour map, a first display field of view (DFOV), acquire diagnostic scan data of the patient according to the one or more scan protocols and parameters, reconstruct the diagnostic scan data according to the first DFOV to generate one or more reconstructed images, and display the one or more reconstructed images on a display device communicably coupled to the imaging system. In a first example of the system, the contour map is generated based on a segmentation mask of one or more scout images. In a second example of the system, optionally including the first example, determining the DFOV comprises determining a widest dimension of the patient based on the contour map. In a third example of the system, optionally including one or both of the first and second examples, the widest dimension is measured as a diameter of a largest circle within an axial plane of the contour map positioned around an isocenter. In a fourth example of the system, optionally including one or more or each of the first through third examples, the imaging system is one of a positron emission tomography (PET) system, a computed tomography (CT) system, a PET-CT system, and a single photon emission computed tomography (SPECT) system. In a fifth example of the system, optionally including one or more or each of the first through fourth examples, the computing device is further configured with instructions that when executed cause the computing device to determine at least one of a noise level and edges within the one or more reconstructed images and output a notification on the display device indicating recommendation for DFOV adjustment. In a sixth example of the system, optionally including one or more or each of the first through fifth examples, the computing device is further configured with instructions that when executed cause the computing device to apply retrospective reconstruction to the diagnostic scan data according to a second DFOV, wherein the second DFOV is larger than the first DFOV.

The disclosure also provides support for a method for determining a display field of view (DFOV), comprising: determining a body contour of a patient based on one or more scout images acquired of the patient, determining the DFOV based on the body contour of the patient, and reconstructing acquired diagnostic scan data of the patient according to the DFOV, wherein the one or more scout images and the diagnostic scan data are acquired by an imaging system and wherein the patient remains positioned within a scanner of the imaging system between acquisition of the one or more scout images and acquisition of the diagnostic scan data. In a first example of the method, the DFOV is further determined based on one or more scan protocols and one or more scan parameters. In a second example of the method, optionally including the first example, determining the body contour of the patient comprises applying a segmentation algorithm to the one or more scout images to generate a contour map and determining a largest dimension of the contour map within an axial plane. In a third example of the method, optionally including one or both of the first and second examples, the largest dimension intersects an isocenter of the one or more scout images. In a fourth example of the method, optionally including one or more or each of the first through third examples, reconstruction of the diagnostic scan data is repeatable with one or more second DFOVs to generate one or more sets of reconstruction images according to a retrospective reconstruction algorithm.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
acquiring one or more scout images of a patient with an imaging system while the patient is positioned within a scanner of the imaging system;
determining a body contour of the patient based on the one or more scout images;
determining a widest dimension of the body contour;
determining, based on the widest dimension, a display field of view (DFOV);
acquiring scan data of the patient with the imaging system;
reconstructing the scan data based on the DFOV to generate one or more reconstructed images; and
displaying the one or more reconstructed images on a display device communicably coupled to the imaging system, wherein the patient remains within the scanner between acquisition of the one or more scout images and the scan data.

2. The method of claim 1, wherein determining the body contour of the patient comprises applying a segmentation mask to generate a contour map.

3. The method of claim 2, wherein determining the widest dimension of the body contour comprises scanning two or more regions of the contour map.

4. The method of claim 1, wherein determining the DFOV is further based on one or more scan protocols and one or more scan parameters.

5. The method of claim 4, wherein the one or more scan protocols comprise a scan type and a scan range.

6. The method of claim 4, wherein the one or more scan parameters comprise a scan field of view (SFOV) and bore size.

7. The method of claim 1, wherein the DFOV is directly related to pixel size of the one or more reconstructed images.

8. The method of claim 1, further comprising applying a retrospective reconstruction algorithm to the scan data based on a second DFOV to generate one or more second reconstructed images in response to detection of one of excess noise and truncated data.

9. A system, comprising:
a computing device communicatively coupled to an imaging system configured to image a patient, the computing device configured with instructions in non-transitory memory that when executed cause the computing device to:
obtain one or more scan protocols and parameters for a requested scan of the patient;
obtain one or more scout images of the patient according to the one or more scan protocols and parameters;
generate a contour map of a body contour of the patient based on the one or more scout images;
determine, based on the contour map, a first display field of view (DFOV);
acquire diagnostic scan data of the patient according to the one or more scan protocols and parameters;
reconstruct the diagnostic scan data according to the first DFOV to generate one or more reconstructed images; and
display the one or more reconstructed images on a display device communicably coupled to the imaging system.

10. The system of claim 9, wherein the contour map is generated based on a segmentation mask of one or more scout images.

11. The system of claim 9, wherein determining the DFOV comprises determining a widest dimension of the patient based on the contour map.

12. The system of claim 11, wherein the widest dimension is measured as a diameter of a largest circle within an axial plane of the contour map positioned around an isocenter.

13. The system of claim 9, wherein the imaging system is one of a positron emission tomography (PET) system, a computed tomography (CT) system, a PET-CT system, and a single photon emission computed tomography (SPECT) system.

14. The system of claim 9, wherein the computing device is further configured with instructions that when executed cause the computing device to determine at least one of a noise level and edges within the one or more reconstructed images and output a notification on the display device indicating recommendation for DFOV adjustment.

15. The system of claim 9, wherein the computing device is further configured with instructions that when executed cause the computing device to apply retrospective reconstruction to the diagnostic scan data according to a second DFOV, wherein the second DFOV is larger than the first DFOV.

16. A method for determining a display field of view (DFOV), comprising:
determining a body contour of a patient based on one or more scout images acquired of the patient;
determining the DFOV based on the body contour of the patient; and
reconstructing acquired diagnostic scan data of the patient according to the DFOV, wherein the one or more scout images and the diagnostic scan data are acquired by an imaging system and wherein the patient remains positioned within a scanner of the imaging system between acquisition of the one or more scout images and acquisition of the diagnostic scan data.

17. The method of claim 16, wherein the DFOV is further determined based on one or more scan protocols and one or more scan parameters.

18. The method of claim 16, wherein determining the body contour of the patient comprises applying a segmentation algorithm to the one or more scout images to generate a contour map and determining a largest dimension of the contour map within an axial plane.

19. The method of claim 18, wherein the largest dimension intersects an isocenter of the one or more scout images.

20. The method of claim 16, wherein reconstruction of the diagnostic scan data is repeatable with one or more second DFOVs to generate one or more sets of reconstruction images according to a retrospective reconstruction algorithm.
